(19) United States
(12) Patent Application Publication
(10) Pub. No.: US 2002/0028917 A1
(43) Pub. Date: Mar. 7, 2002

(54) Separation of avian antibodies

(76) Inventors: Sarah Charlotte Gee, Belrose (AU); Irene Bate, Dee Why (AU); Theresa Marie Thomas, Schofields (AU); Dennis Brian Rylatt, Ryde (AU)

Correspondence Address: ARTER & HADDEN, LLP, 1100 HUNTINGTON BUILDING, 925 EUCLID AVENUE, CLEVELAND, OH, 44115-1475, US

(21) Appl. No.: 09/853,814

(22) Filed: May 11, 2001

(30) Foreign Application Priority Data
May 11, 2000 (AU) PQ7433

(51) Int. Cl. C07K 16/00

(52) U.S. Cl. 530/388.1

(57) ABSTRACT

A method for obtaining avian antibodies from eggs, the method comprising the steps of:

(a) providing egg material containing avian antibodies;

(b) adjusting the pH of the egg material such that the antibodies in the sample have a desired charge;

(c) placing the pH adjusted egg material in a first interstitial volume or solvent stream, the first interstitial volume being separated from a second interstitial volume or solvent stream by an electrophoretic separation barrier membrane having a molecular mass cut-off greater than the molecular mass of avian antibodies, the second interstitial volume comprising a solvent with a desired pH;

(d) applying a voltage potential between the first and second interstitial volumes causing avian antibodies to move through the electrophoretic separation barrier into the second interstitial volume while other components in the egg material are substantially retained in the first interstitial volume, or if entering the electrophoretic separation barrier, being substantially prevented from passing through the electrophoretic separation barrier to the second interstitial volume; and

(e) maintaining steps (d) until the desired amount of avian antibodies has been obtained from the egg material.

### BACKGROUND OF THE INVENTION

The present invention relates to methods for the obtaining or separation of avian antibodies, particularly methods for obtaining antibodies from chicken eggs.

Avian antibodies, particularly chicken immunoglobulins (IgY), are becoming increasingly useful tools for the construction of immunoassays. IgY is structurally similar to mammalian immunoglobulins (IgG). The antibody is approximately 180 kDa and has two heavy chains (“nu”) and two light chains. Whilst functionally equivalent to IgG, IgY is immunologically different as the nu chains lack Fc domains. As a result, there is reduced cross-reactivity in immunoassays, and the larger evolutionary distance from mammals increases the possibility of producing antibodies to the highly conserved mammalian proteins (e.g. insulin and cytochrome C). The specific antibodies IgY developed in chickens are therefore likely to have high avidity to mammalian antigens.

Other advantages include a lack of reactivity to rheumatoid factors and the inability to activate complement components in mammalian sera. A further advantage of avian antibodies is that they are more resistant to extremes of temperature, pI and ionic strength.

A single egg contains as much antibody as an average bleed from a rabbit. In addition, egg collection is simple and non-invasive. Eggs are generally laid on a daily basis, whereas a rabbit may only be bled once every 2-3 weeks. Furthermore, the quantity of IgY in one egg yolk is appreciable (easily 50-100 mg) and the hen can lay one egg per day. The birds are easy to feed in comparison with mammalian animals (rabbits, sheep, goats etc), resulting in lower production costs.

Chickens typically require only a one month immunization regimen to obtain high titers of polyclonal antibody. To ensure a rabbit is producing high titers of antibody, the immunization procedure typically requires up to 2-3 months. Also, the response for specific antibody in birds is much quicker than in mammalian animals. Antibodies are produced is sufficient amounts and obtainable from the 30th day post immunization in chickens compared with from around the 60th day in rabbits and longer in larger mammalian animals.

Unfortunately, separation or purification of antibodies from eggs has proved more difficult than for mammalian antibodies. The most commonly used and convenient method of purification for conventional mammalian antibodies is affinity chromatography on Protein A- or Protein G-bound supports. This method is not applicable as avian antibodies do not bind bacterial or mammalian Fc receptors. Other methods, such as chemical precipitation or size exclusion chromatography, are not particularly suitable for commercial production as the methods are complicated, expensive and time consuming. There is a real need for improved methods for the separation of avian antibodies, particularly in commercial quantities.

The present inventors have now developed methods for separating avian antibodies using electrophoretic separation that are fast and result in high yields of antibodies.

### SUMMARY OF THE INVENTION

In accordance with the present invention, there is provided a method and apparatus for separating avian antibodies using electrophoretic separation which are fast and result in high yields of antibodies.

Further, in accordance with the present invention, there is provided a method and apparatus for separating avian antibodies which removes potentially infectious or pathogenic contaminants or agents from the antibody sample or egg material. As eggs are a potential source of viruses, it is possible to ensure removal of any contaminating viral material during electrophoresis. Cross-species transfer of pathogens is occurring at an increased rate so the ability to ensure that viruses or bacteria can be removed from an egg sample is an advantage over prior art methods.

Still further, in accordance with the present invention, there is provided a method and apparatus for separating avian antibodies which results in yields of avian antibodies of at least 70% with a purity of at least 90% from egg samples.

Still further, in accordance with the present invention, there is provided a method for obtaining avian antibodies from eggs, the method comprising the steps of:

(a) providing egg material containing avian antibodies;

(b) adjusting the pH of the egg material, if required, such that the antibodies in the sample have a desired charge;

(c) placing the pH-adjusted egg material in a first interstitial volume or solvent stream, the first interstitial volume being separated from a second interstitial volume or solvent stream by an electrophoretic separation barrier or membrane having a molecular mass cut-off greater than the molecular mass of avian antibodies, the second interstitial volume comprising a solvent with a desired pH;

(d) applying a voltage potential between the first and second interstitial volumes causing avian antibodies to move through the electrophoretic separation barrier into the second interstitial volume while other components in the egg material are substantially retained in the first interstitial volume, or if entering the electrophoresis separation membrane, being substantially prevented from passing through the electrophoretic separation barrier to the second interstitial volume;

(e) optionally, periodically stopping and reversing the voltage potential to cause movement of components having entered the electrophoretic separation barrier to move back into the first interstitial volume, wherein substantially not causing any antibodies having entered the second interstitial volume to re-enter first interstitial volume;

(f) optionally, periodically removing the second interstitial volume containing antibodies and replacing the second interstitial volume with fresh solvent;

(g) maintaining step (d), and optionally steps (e) and (f), until the desired amount of avian antibodies has been obtained from the egg material.

Still further in accordance with the present invention, there is provided an apparatus for separating avian antibodies, the apparatus comprising:

(a) a cathode in a cathode zone;

(b) an anode in an anode zone, the anode disposed relative to the cathode so as to be adapted to generate an electric field in an electric field area therebetween upon application of a voltage potential between the cathode and the anode;

(c) an electrophoretic separation barrier or membrane having a defined pore size disposed in the electric field area;

(d) a first restriction barrier or membrane disposed between the cathode zone and the electrophoretic separation barrier so as to define a first interstitial volume therebetween;

(e) a second restriction barrier or membrane disposed between the anode zone and the electrophoretic separation barrier so as to define a second interstitial volume therebetween;

(f) means adapted to provide solvent to the cathode zone, the anode zone and at least one of the first and second interstitial volumes;

(g) means adapted to provide a sample constituent in a selected one of the first and second interstitial volumes wherein upon application of the voltage potential, a compound in the sample is moved through at least one barrier and provided to the other of the first and second interstitial volumes or the cathode or anode zones.

An advantage of the method and apparatus for separating avian antibodies according to the present invention is that such separation removes potentially infectious or pathogenic contaminants or agents from the antibody sample or egg material.

Another advantage of the method and apparatus for separating avian antibodies according to the present invention is that such separation results in yields of avian antibodies of at least 70% with a purity of at least 90% from egg samples.

These and other advantages will be apparent to one skilled in the art upon reading and understanding the specification.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

### BRIEF DESCRIPTION OF THE DRAWINGS

FIG. 1 shows a polyacrylamide gel analysis of separation of chicken antibodies according to a preferred method of present invention. The egg sample was placed in a first solvent stream positioned near the anode region of an electrophoresis apparatus and antibodies were caused to move toward the cathode region of the apparatus into a second solvent stream. Majority of egg proteins remained in the sample while the antibody was selectively moved to the second solvent stream for collection. (Well 1: 0 time egg sample; Well 2: 30 minutes second stream; Well 3: 60 minutes second stream; Well 4: 90 minutes second stream; Well 5: 120 minutes second stream; Well 6: PBS wash second stream; Well 7: 90 minutes first stream; Well 8: 120 minutes first stream; Well 9: PBS wash first stream; Well 10: molecular mass markers).

### DETAILED DESCRIPTION OF THE INVENTION

In a first aspect of the present invention, there is provided a method for obtaining avian antibodies from eggs, the method comprising the steps of:

(a) providing egg material containing avian antibodies;

(b) adjusting the pH of the egg material, if required, such that the antibodies in the sample have a desired charge;

(c) placing the pH-adjusted egg material in a first interstitial volume or solvent stream, the first interstitial volume being separated from a second interstitial volume or solvent stream by an electrophoretic separation barrier or membrane having a molecular mass cut-off greater than the molecular mass of avian antibodies, the second solvent stream comprising a solvent with a desired pH;

(d) applying a voltage potential between the first and second interstitial volumes causing avian antibodies to move through the electrophoretic separation barrier into the second interstitial volume while other components in the egg material are substantially retained in the first interstitial volume, or if entering the electrophoretic separation barrier, being substantially prevented from passing through the electrophoretic separation barrier to the second interstitial volume;

(e) optionally, periodically stopping and reversing the voltage potential to cause movement of components having entered the electrophoretic separation barrier to move back into the first interstitial volume, wherein substantially not causing any antibodies having entered the second interstitial volume to re-enter first interstitial volume;

(f) optionally, periodically removing the second interstitial volume containing antibodies and replacing the second interstitial volume with fresh solvent;

(g) maintaining step (d), and optionally steps (e) and (f), until the desired amount of avian antibodies has been obtained from the egg material.

Preferably, the avian antibodies are chicken antibodies.

Preferably, the egg material is egg yolk separated from egg whites and diluted in a suitable buffer. As egg yolk is very viscous and proteinaceous, dilution has been found to be useful to enhance the degree or yield of antibody separation. A dilution of at least about 1:2, preferably about 1:5, and more preferably about 1:10 or greater has been found to be suitable for the present invention. The egg yolks are diluted in any suitable buffer or solvent having the required pH. A pH of around 7 or less has been found to be suitable to obtain high yields of relatively pure antibodies. Examples of diluting buffers include, but are not limited to, phosphate buffered saline, Tris, Mes-Histidine pH 5.5 (Mes is an abbreviation for 2-[N-Morphilino]ethanesulphonic acid) and Mes-Bis-Tris pH 6.0 (Bis-Tris is an abbreviation for (bis[2-Hydroxethyl]iminotris[hydroxymethyl]methane; 2-bis[2-Hydroxyethyl]amino-2-[hydroxymethyl]-1,3 -propanediol). It will be appreciated, however, that other buffers or solvents are suitably used to dilute egg material.

For large scale separation, the egg yolks are pooled from a number of eggs. If a large number of birds have been immunised against the same antigen, then eggs can be pooled from a number of birds. Alternatively, eggs laid by the one immunised bird are collected and pooled to ensure knowledge of the origin of the antibodies.

In order to assist in the yields of antibodies, it is preferred that the yolks are separated cleanly from egg whites. By removing egg white material before subjecting the egg material to electrophoresis, potentially contaminating components from egg whites will not be present in the starting material. In order to assist in the preparation of the egg material, the separated yolks are diluted and centrifuged to remove any precipitated material. In order to assist in the separation of the antibodies, the egg yolks are preferably broken up to form a liquid, usually by suitable mechanical means.

In one preferred form, step (b) results in the antibodies having a positive charge and the antibodies are caused to move toward the cathode of an electrophoretic apparatus used for the method. A pH of about 6 or less, preferably around 5.5, has been determined to be suitable in this regard as other components in the egg material are negatively charged and do not move with the antibodies to the second solvent stream. Due to the complex mixture of compounds in egg material, it would not be expected that such high yields of relatively pure antibodies could be produced using electrophoretic means.

Any buffer or solvent having the desired pH is suitable for the second solvent stream. The buffer Mes-Histidine pH 5.5, has been found to be suitable for some separations. Other buffers such as phosphate buffered saline, Tris, Mes-Histidine (Mes is an abbreviation for 2-[N-Morphilino]ethanesulphonic acid) and Mes-Bis-Tris (Bis-Tris is an abbreviation for (bis[2-Hydroxethyl]iminotris[hydroxymethyl]methane; 2-bis[2-Hydroxyethyl]amino-2-[hydroxymethyl]-1,3 -propanediol) are also applicable.

In one embodiment, the solutions or solvents in the first and second solvent streams are cooled by any suitable means to ensure little or no inactivation of the antibodies occurs during the separation process and to maintain a desired temperature of the materials during separation.

The electrophoretic separation barrier or membrane is preferably comprised of polyacrylamide and having a defined molecular mass cut-off. Preferably, the electrophoretic separation membrane has a molecular mass cut-off up to about 2000 kDa. It will be appreciated, however, that other membrane chemistries or constituents are suitable for the present invention.

A molecular mass cut off of about 1500 kDa has been found to be particularly suitable for the electrophoretic separation membrane. It will be appreciated, however, that other cut-off membranes that allow the movement of avian antibodies are also suitable.

The restriction barriers or membranes are preferably formed from polyacrylamide and having a molecular mass cut-off less than the electrophoretic separation membrane, preferably from about 1 kDa to about 150 kDa. The selection of the molecular mass cut-off of the restriction membranes will depend on the sample being processed and other materials present in the sample.

In a preferred form, the electrophoretic separation membrane forms part of a separation cartridge where two restriction membranes having molecular mass cut-offs less than the electrophoretic separation membrane are positioned and spaced either side of the electrophoretic separation membrane thus forming the first and second solvent streams. An asymmetrical arrangement with a 150 kDa restriction membrane and a 5 kDa restriction membrane positioned either side of a 1500 kDa separation membrane has been found to be particularly suitable for the present invention. It will be appreciated, however, that membrane configurations where both restriction membranes have the same molecular mass cut-off, such as 150 kDa, are also suitable.

Selection or application of the voltage and/or current applied varies depending on the separation. Typically up to several thousand volts are used but choice and variation of voltage will depend on the configuration of the apparatus, buffers and the sample to be separated. In a laboratory scale instrument, the preferred voltage is about 250 V. However, depending on transfer, efficiency, scale-up and particular method from about 0 V to about 5000 V are used. Higher voltages are also considered, depending on the apparatus and sample to be treated.

Optionally, the voltage potential is periodically stopped and reversed to cause movement of a constituent having entered a membrane to move back into the volume or stream from which it came, while substantially not causing any constituents that have passed completely through a membrane to pass back through the membrane.

Reversal of current is an option but another alternative or adjunct is a resting period. Resting (a period without an electric potential being applied) is an optional step that can replace or be included before or after or instead of an optional voltage potential reversal. This resting technique is often practised for specific separation applications as an alternative or adjunct to reversing the potential. Alternatively a wash with a buffer or solution of high or physiological salt concentration (for example phosphate buffered saline) is circulated through the first and/or second solvent streams.

Preferably a voltage potential of about 200 V to about 250 V and current of about 500 mA is applied to give a separation run in about 2 hours. Other voltages and currents, higher or lower, are also suitable for the present invention depending on the separation membrane used, the volume or concentration of egg yolk to be processed, and the speed of separation required. It is understood, however, that run times may vary from 30 minutes to 4 to 5 hours or longer depending on the conditions.

Step (f), if used, is usually determined by the amount of antibodies present in the second solvent stream. Thirty minute intervals have been found to be suitable but this varies depending on the rate of movement into the second solvent stream or the amount of egg material being processed.

Some benefits of the method according to the first aspect of the present invention are the possibility of scale-up without adversely altering the properties of the avian antibodies being separated.

Another benefit of the present invention is that it is possible to remove potentially A infectious or pathogenic contaminants or agents from the antibody sample or egg material. As eggs are a potential source of viruses, it is possible to ensure removal of any contaminating viral material during electrophoresis. Cross-species transfer of pathogens is occurring at an increased rate so the ability to ensure that viruses or bacteria can be removed from an egg sample is an advantage over prior art methods.

Due to the viscous nature of eggs and the large amounts of complex proteins present, electrophoresis methods would not typically be considered as suitable candidates for use in separating avian antibodies. Surprisingly, the present inventors have been able to modify electrophoresis methods to obtain unexpectedly good yields of high purity antibodies.

The method according to the present invention results in yields of avian antibodies of at least 70% with a purity of at least 90% from egg samples.

The method according to the present invention results in substantially purified or isolated avian antibodies from eggs in only several hours compared with up to several days using prior art affinity or precipitation methods. The speed of separation and purity of the final product provides a great advance over current methods. Not only does the method according to the present invention allow the processing of large volumes of eggs, the method is fast and extremely efficient.

Flow rate of sample/buffer/fluid has an influence on the separation of antibodies and removal of other components in the egg sample. Rates of millilitres per hour up to litres per hour are used depending on the configuration of the apparatus and the sample to be separated. Currently in a laboratory scale instrument, the preferred flow rate is about 20±5 mL/min. However, flow rates ranging from about 0 mL/min to about 50,000 mL/min are used across the various separation regimes. The maximum flow rate is even higher, depending on the pumping means and size of the apparatus. The selection of the flow rate is dependent on the product to be transferred, efficiency of transfer, pre- and post-positioning with other applications.

In one preferred method, step (a) comprises the steps:

(i) separating egg yolk from egg white;

(ii) mixing the egg yolk until liquid;

(iii) diluting egg yolk in buffer with a desired pH; and

(iv) removing any precipitate from the diluted egg yolk.

Preferably, the desired buffer is PBS (phosphate buffered saline) about pH 7 or less.

Preferably, step (iv) is achieved by centrifuging the diluted egg yolk.

In another preferred method, step (a) comprises the steps:

(i) separating egg yolk from egg white;

(ii) mixing the egg yolk until liquid;

(iii) diluting egg yolk in water;

(iv) raising the pH of the diluted egg yolk to about 7 or less;

(v) leaving the pH-adjusted diluted egg yolk to stand at a cool temperature for a desired period; and

(vi) removing any precipitate from the diluted egg yolk.

Preferably, the pH of the diluted egg yolk mixture is raised to about 7 with 1M TRIS and the pH-adjusted diluted egg yolk mixture allowed to stand at about 4° C. overnight.

Preferably, step (vi) is achieved by centrifuging the diluted egg yolk.

Preferably, the antibodies are removed from egg material using an electrophoretic apparatus comprising:

(a) a cathode in a cathode zone;

(b) an anode in an anode zone, the anode disposed relative to the cathode so as to be adapted to generate an electric field in an electric field area therebetween upon application of a voltage potential between the cathode and the anode;

(c) an electrophoretic separation barrier membrane having a defined pore size disposed in the electric field area;

(d) a first restriction barrier or membrane disposed between the cathode zone and the electrophoretic separation barrier so as to define a first interstitial volume or solvent stream therebetween;

(e) a second restriction barrier or membrane disposed between the anode zone and the electrophoretic separation barrier so as to define a second interstitial volume or solvent stream therebetween;

(f) means adapted to provide solvent to the cathode zone, the anode zone and at least one of the first and second interstitial volumes;

(g) means adapted to provide a sample constituent in a selected one of the first and second interstitial volumes wherein upon application of the voltage potential, a compound in the sample is moved through at least one barrier and provided to the other of the first and second interstitial volumes or the cathode or anode zones.

The cathode zone and the anode zone are supplied with suitable buffer solutions by any suitable pumping means. A sample to be processed is supplied directly to the first or second interstitial volumes by any suitable pumping means.

Preferably, the zones and the first and second interstitial volumes are configured to allow flow of the respective fluid/buffer and sample solutions forming streams. In this form, large volumes can be processed quickly and efficiently. The solutions are typically moved or recirculated through the zones and volumes from respective reservoirs by suitable pumping means. In a preferred embodiment, peristaltic pumps are used as the pumping means for moving the sample, buffers or fluids.

In one embodiment, the buffer and sample solutions are cooled by any suitable means to ensure no inactivation of antibodies occurs during the separation process and to maintain a desired temperature of the apparatus while in use.

Preferably, in order to collect and/or concentrate separated constituents, solution in at least one of the streams containing any separated compounds or molecules is collected and replaced with suitable solvent to ensure that electrophoresis can continue in an efficient manner.

In a preferred method wherein the antibodies have a positive charge, a sample in the form of egg material is placed in the second solvent stream, buffer or solvent is provided to the electrode zones and the first solvent stream, a voltage potential is applied to the electric field area causing antibodies in the sample to move to buffer in the first solvent stream while other components in the egg material remain in the second solvent stream.

It will be appreciated that this procedure can be reversed where the samples in placed in the first solvent stream, the polarity of the electrodes being reversed so the product is collected in the second solvent. In some applications the sample is placed in either the first or second solvent stream and the impurities removed to the opposite stream.

In a second aspect, the present invention provides avian antibodies obtained by the method according to the first aspect of the present invention.

In a third aspect, the present invention provides use of avian antibodies according to the second aspect of the present invention in scientific, medical and veterinary applications. Typically, the antibodies are used in immunoassays where birds, preferably chickens, are immunised with required antigens to generate the required antibodies.

In a fourth aspect, the present invention consists in substantially pure isolated antibodies from avian eggs, preferably antibodies from chicken eggs, having immunoactivity mimicking antibodies in their natural state.

In order that the present invention may be more clearly understood, preferred forms will be described with reference to the following drawing and examples.

Egg Sample Preparation

Method 1

Separate egg yolk from the egg white.

Mix the egg yolk until liquid.

Dilute 5 mL of egg yolk in 45 mL of water.

Place the mixture on a stirrer and raise the pH to about 7 with 1M TRIS.

Refrigerate overnight.

Centrifuge at 10,000 g for about 30 minutes at about 4 degrees Celsius.

Discard the pellet, retain the supernatant.

Subject egg sample to electrophoresis to obtain antibodies.

Method 2

Separate egg yolk from the egg white.

Mix the egg yolk until liquid.

Dilute 5 mL of egg yolk in 45 mL of PBS (phosphate buffered saline) at about pH 7.

Centrifuge at 10,000 g for about 45 minutes at 4° C.

Filter supernatant through polyethylene paper, discard the pellet.

Antibody Separation Example 1

An electrophoresis apparatus produced by Gradipore Limited, Australia was used. An electrophoresis separation cartridge, adapted to be used in the electrophoresis apparatus with a 150 kDa upper restriction membrane, a large pore sized separation membrane (˜1500 kDa) and a 5 kDa lower restriction membrane was used. The upper restriction membrane separates the cathode zone from the second solvent stream and the lower restriction membrane separates the anode zone from the first solvent stream in an electrophoresis apparatus.

MES/histidine pH 5.5 buffer (40 mM) was used as the electrophoresis buffer, the second solvent stream to which the antibodies move during electrophoresis, and for the dilution of the egg sample.

Ten mL of egg yolk supernatant was diluted in 10 mL of MES/histidine pH 5.5 buffer.

Twenty mL of MES/histidine pH 5.5 (100 mM) was used for passing through the second solvent stream.

Egg sample was placed in a first solvent stream which was adjacent the anode zone of an electrophoresis apparatus.

As the antibodies had a positive charge at the pH selected, during the influence of an electric field or voltage potential, the antibodies were caused to move toward the cathode through the separation membrane into the second solvent stream for collection. Other components in the egg material remained in the first solvent stream as they could not pass the electrophoresis membrane.

Electrophoresis was run for two hours at 250V.

Liquid from the first solvent stream and the second solvent stream were removed at the conclusion of the electrophoresis run.

Ten mL of PBS was flushed through solvent streams. The PBS was circulated with no current for 5 minutes (to collect any remaining antibody from the tubing and/or the membranes).

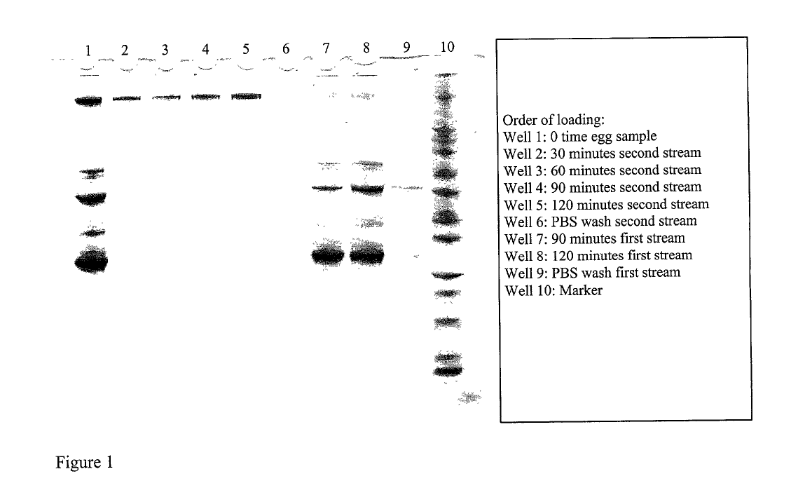

Table 1 and FIG. 1 show the results of a separation of avian antibodies using egg sample preparation method 2. As can be seen from the results, over 82% total recovery was achieved. After only 30 minutes, relatively large amounts of antibody were caused to move out of the egg sample material to the second solvent stream while other unwanted components from the egg material remained in the sample.

TABLE 1
Enzyme Immunoassay for Avian Antibody

| | Volume | Antibody | Total | Recovery |
|---|---|---|---|---|
| Sample | (mL) | (mg/mL) | antibody (mg) | (%) |
| 0 time* | 20 | 0.32 | 6.2 | |
| first stream (final) | 17.5 | 0.26 | 4.6 | 74.2 |
| first stream (wash) | 10 | 0.05 | 0.5 | 8.0 |
| Total | | | | 82.2 |

*starting egg material

Antibody Separation Example 2

Egg Sample Preparation

Yolk was separated from the egg white while being careful to ensure that no white was in the final yolk. The yolk was broken up so it became a liquid.

Eighteen mL of distilled water was added to a glass beaker and mixed on a magnetic stirrer. Two mL of the egg yolk was added to stirring water and mixed until combined. The mixed yolk was adjusted to pH 7 with 1M Tris and left overnight at 4° C.

The yolk mixture was centrifuged at 10,000 g at 4° C. for 30 minutes (Biofuge Stratus from Heraeus) the remove particulate matter. The egg yolk supernatant was collected and the pellet discarded.

Antibody Separation

An electrophoresis apparatus produced by Gradipore Limited, Australia was used. An electrophoresis separation cartridge, adapted to be used in the electrophoresis apparatus with a 100 kDa upper restriction membrane, a large pore sized separation membrane (˜1500 kDa) and a 100 kDa lower restriction membrane was used. The upper restriction membrane separates the cathode zone from the second solvent stream and the lower restriction membrane separates the anode zone from the first solvent stream in an electrophoresis apparatus.

Buffer, 31 mM Mes/Bis-Tris pH 6.0 buffer by mixing 9 g Bis-Tris and 17 g Mes in 4 L of distilled water was prepared.

Ten mL of the egg yolk supernatant was added to the first stream adjacent the anode zone of the electrophoresis apparatus and 10 mL of Mes/Bis-Tris buffer placed in the second solvent stream adjacent the cathode zone of the apparatus.

Voltage was set at 200 V and current at 500 mA and the separation was started by switching on a power source and pumping the material respective streams.

At 30 minutes, the second solvent stream containing the antibodies was harvested and replaced with fresh 10 mL Mes/Bis-Tris buffer. The running or back buffer was replaced with the remaining 2 L of prepared buffer.

The separation was continued for a further 90 minutes, harvesting the second stream every 30 minutes.

As the antibodies had a positive charge at the pH selected, during the influence of an electric field or voltage potential, the antibodies were caused to move toward the cathode through the separation membrane into the second solvent stream for collection. Other components in the egg material remained in the first solvent stream as they could not pass the electrophoresis membrane.

The process yielded more than 70% of total antibodies present in the egg yolk sample in a relatively pure state.

The present inventors have found that IgY can be purified in a single electrophoresis run. The purification/separation method according to the present invention should make large quantities of avian antibodies more available and their use more acceptable.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive. Other features and aspects of this invention will be appreciated by those skilled in the art upon reading and comprehending this disclosure. Such features, aspects, and expected variations and modifications of the reported results and examples are clearly within the scope of the invention where the invention is limited solely by the scope of the following claims.

What we claim is:

1. A method for obtaining avian antibodies from eggs, the method comprising the steps of:
(a) providing egg material containing avian antibodies;
(b) adjusting the pH of the egg material such that the antibodies in the sample have a desired charge;
(c) placing the pH-adjusted egg material in a first interstitial volume, the first interstitial volume being separated from a second interstitial volume by an electrophoretic separation barrier having a molecular mass cut-off greater than the molecular mass of avian antibodies, the second interstitial volume comprising a solvent with a desired pH;
(d) applying a voltage potential between the first and second interstitial volumes causing avian antibodies to move through the electrophoretic separation barrier into the second interstitial volume while other components in the egg material are substantially retained in the first interstitial volume, or if entering the electrophoretic separation barrier, being substantially prevented from passing through the electrophoretic separation barrier to the second interstitial volume; and
(e) maintaining step (d) until the desired amount of avian antibodies has been obtained from the egg material.

2. The method according to claim 1 further comprising:
(f) periodically stopping and reversing the voltage potential to cause movement of components having entered the electrophoretic separation barrier to move back into the first interstitial volume, wherein substantially not causing any antibodies having entered the second interstitial volume to re-enter first interstitial volume.

3. The method according to claim 2 further comprising:
(g) periodically removing the second interstitial volume containing antibodies and replacing the second interstitial volume with fresh solvent.

4. The method according to claim 1 wherein the avian antibodies are chicken antibodies.

5. The method according to claim 1 wherein the egg material is egg yolk separated from egg whites, broken up and diluted.

6. The method according to claim 5 wherein the separated egg yolk is diluted at least about 1:2.

7. The method according to claim 5 wherein the separated egg yolk is diluted at least about 1:5.

8. The method according to claim 5 wherein the separated egg yolk is diluted at least about 1:10.

9. The method according to claim 5 wherein the egg yolk is diluted in a buffer selected from the group consisting of water, phosphate buffered saline, Tris, Mes-Histidine, Mes-Bis-Tris, and combinations thereof.

10. The method according to claim 1 wherein pH adjustment step (b) results in the antibodies having a positive charge and the antibodies are caused to move from the egg sample toward a cathode of an electrophoresis apparatus during electrophoresis.

11. The method according to claim 10 wherein the egg sample is adjusted to a pH up to about 6.

12. The method according to claim 10 wherein the egg sample is adjusted to a pH up to about 5.5.

13. The method according to claim 10 wherein the pH is adjusted using a buffer selected from the group consisting of Mes-histidine buffer, Mes/Bis-Tris buffer, Tris, acetic acid, and combinations thereof.

14. The method according to claim 1 wherein the electrophoretic separation barrier is a membrane comprised of polyacrylamide.

15. The method according to claim 14 wherein the electrophoretic separation membrane has a molecule mass cut-off up to about 1500 kDa.

16. The method according to claim 1 wherein step (d) is carried out at a voltage of up to about 250 V.

17. The method according to claim 1 wherein yields of avian antibodies of at least about 70% with a purity of at least about 90% from egg samples are achieved.

18. The method according to claim 1 wherein step (a) comprises the steps:
(i) separating egg yolk from egg white;
(ii) mixing the egg yolk until liquid;
(iii) diluting egg yolk in buffer with a desired pH; and
(iv) removing any precipitate from the diluted egg yolk.

19. The method according claim 18 wherein the buffer is PBS (phosphate buffered saline) pH 7.

20. The method according claim 18 wherein step (iv) is achieved by centrifuging the diluted egg yolk.

21. The method according to claim 1 wherein step (a) comprises the steps:
(i) separating egg yolk from egg white;
(ii) mixing the egg yolk until liquid;
(iii) diluting egg yolk in water;
(iv) raising the pH of the diluted egg yolk mixture to about 7;
(v) leaving the pH-adjusted diluted egg yolk mixture to stand at a cool temperature for a desired period; and
(vi) removing any precipitate from the pH-adjusted diluted egg yolk.

22. The method according to claim 21 wherein the pH of the diluted egg yolk mixture is raised to about 7 with 1M TRIS and the pH-adjusted diluted egg yolk mixture allowed to stand at about 4° C. overnight.

23. The method according claim 22 wherein step (vi) is achieved by centrifuging the diluted egg yolk.

24. An isolated avian antibody obtained by the method according to claim 1.

25. The avian antibodies according to claim 24 comprising at least about 90% purity.

26. The avian antibodies according to claim 24 wherein the antibodies are used in scientific, medical and veterinary applications.

27. The avian antibodies according to claim 24 wherein the antibodies are used in immunoassays

28. An electrophoretic separation apparatus for separating avian antibodies, the apparatus comprising:
(a) a cathode in a cathode zone;
(b) an anode in an anode zone, the anode disposed relative to the cathode so as to be adapted to generate an electric field in an electric field area therebetween upon application of a voltage potential between the cathode and the anode;
(c) an electrophoretic separation barrier having a defined pore size disposed in the electric field area;
(d) a first restriction barrier disposed between the cathode zone and the electrophoretic separation barrier so as to define a first interstitial volume therebetween;
(e) a second restriction barrier disposed between the anode zone and the electrophoretic separation barrier so as to define a second interstitial volume therebetween;
(f) means adapted to provide solvent to the cathode zone, the anode zone and at least one of the first and second interstitial volumes;
(g) means adapted to provide a sample constituent comprising avian antibodies in a selected one of the first and second interstitial volumes wherein upon application of the voltage potential, selected separation products in the sample are moved through at least one barrier and provided to the other of the first and second interstitial volumes or the cathode or anode zones.

29. The apparatus according to claim 28 wherein the apparatus further comprises means to periodically stop and reverse the voltage potential to cause movement of selected compounds having entered the electrophoretic separation barrier to move back into the selected one of the interstitial volume into which the sample was originally provided, wherein substantially not causing any of the selected separation products having entered the other of the interstitial volumes to re-enter the interstitial volume into which the sample was originally provided.

30. The apparatus according to claim 29 wherein the apparatus further comprises means to periodically remove the interstitial volume containing the selected separation product and replacing such interstitial volume with fresh solvent.

31. The apparatus according to claim 28 wherein the avian antibodies are chicken antibodies.

32. The apparatus according to claim 28 wherein the sample constituent is egg material

33. The apparatus according to claim 32 wherein the egg material is egg yolk separated from egg whites, broken up and diluted.

34. The apparatus according to claim 34 wherein the separated egg yolk is diluted at least about 1:2.

35. The apparatus according to claim 28 wherein yields of avian antibodies of at least about 70% with a purity of at least about 90% from samples are achieved
